SEQUENCE LISTING (33 sequences)

SEQ ID NO: 1 - protein, 7 aa, Artificial sequence
  synthetic
SNYLYDN

SEQ ID NO: 2 - protein, 9 aa, Artificial sequence
  synthetic
REFPFYGDY

SEQ ID NO: 3 - protein, 6 aa, Artificial sequence
  synthetic
EKHHSH

SEQ ID NO: 4 - protein, 38 aa, Artificial sequence
  synthetic
DSHEKRHHGYRRKFHEKHHSHREFPFYGDYGSNYLYDN

SEQ ID NO: 5 - protein, 27 aa, Artificial sequence
  synthetic
RKFHEKHHSHREFPFYGDYGSNYLYDN

SEQ ID NO: 6 - protein, 32 aa, Artificial sequence
  synthetic
DSHAKRHHGYKRKFHEKHHSHRGYRSNYLYDN

SEQ ID NO: 7 - protein, 15 aa, Artificial sequence
  synthetic
EKHHSHREFPFYGDY

SEQ ID NO: 8 - protein, 17 aa, Artificial sequence
  synthetic
REFPFYGDYGSNYLYDN

SEQ ID NO: 9 - protein, 19 aa, Artificial sequence
  synthetic
SHREFPFYGDYGSNYLYDN

SEQ ID NO: 10 - protein, 21 aa, Artificial sequence
  synthetic
HHSHREFPFYGDYGSNYLYDN

SEQ ID NO: 11 - protein, 23 aa, Artificial sequence
  synthetic
EKHHSHREFPFYGDYGSNYLYDN

SEQ ID NO: 12 - protein, 25 aa, Artificial sequence
  synthetic
FHEKHHSHREFPFYGDYGSNYLYDN

SEQ ID NO: 13 - protein, 19 aa, Artificial sequence
  synthetic
RKFHEKHHSHREFPFYGDY

SEQ ID NO: 14 - protein, 15 aa, Artificial sequence
  synthetic
EKHHSHREFPFYGDY

SEQ ID NO: 15 - protein, 21 aa, Artificial sequence
  synthetic
RKFHEKHHSHREFPFYGDYGS

SEQ ID NO: 16 - protein, 17 aa, Artificial sequence
  synthetic
EKHHSHREFPFYGDYGS

SEQ ID NO: 17 - protein, 23 aa, Artificial sequence
  synthetic
RKFHEKHHSHREFPFYGDYGSNY

SEQ ID NO: 18 - protein, 19 aa, Artificial sequence
  synthetic
EKHHSHREFPFYGDYGSNY

SEQ ID NO: 19 - protein, 25 aa, Artificial sequence
  synthetic
RKFHEKHHSHREFPFYGDYGSNYLY

SEQ ID NO: 20 - protein, 21 aa, Artificial sequence
  synthetic
EKHHSHREFPFYGDYGSNYLY

SEQ ID NO: 21 - protein, 17 aa, Artificial sequence
  synthetic
RKFHEKHHSHREFPFYG

SEQ ID NO: 22 - protein, 13 aa, Artificial sequence
  synthetic
EKHHSHREFPFYG

SEQ ID NO: 23 - protein, 19 aa, Artificial sequence
  synthetic
HHSHREFPFYGDYGSNYLY

SEQ ID NO: 24 - protein, 17 aa, Artificial sequence
  synthetic
SHREFPFYGDYGSNYLY

SEQ ID NO: 25 - protein, 15 aa, Artificial sequence
  synthetic
SHREFPFYGDYGSNY

SEQ ID NO: 26 - protein, 15 aa, Artificial sequence
  synthetic
FPFYGDYGSNYLYDN

SEQ ID NO: 27 - protein, 13 aa, Artificial sequence
  synthetic
FYGDYGSNYLYDN

SEQ ID NO: 28 - protein, 15 aa, Artificial sequence
  synthetic
HHSHREFPFYGDYGS

SEQ ID NO: 29 - protein, 13 aa, Artificial sequence
  synthetic
SHREFPFYGDYGS

SEQ ID NO: 30 - protein, 24 aa, Artificial sequence
  synthetic
DSHAKRHHGYKRKFHEKHHSHRGY

SEQ ID NO: 31 - protein, 14 aa, Artificial sequence
  synthetic
GYKRKFHEKHHSHR

SEQ ID NO: 32 - protein, 11 aa, Artificial sequence
  synthetic
SHREFPFYGDY

SEQ ID NO: 33 - protein, 44 aa, Artificial sequence
  synthetic
GGDSHEKRHHGYRRKFHEKHHSHREFPFYGDYGSNYLYDNLPET

(19) United States
(12) Patent Application Publication
(10) Pub. No.: US 2017/0232064 A1
(43) Pub. Date: Aug. 17, 2017

(54) FORMULATIONS FOR HISTATIN THERAPEUTICS

(71) Applicant: RAPID PATHOGEN SCREENING, INC., Sarasota, FL (US)

(72) Inventors: Robert P. Sambursky, Bradenton, FL (US); Robert W. Vandine, Montoursville, PA (US); Peter Condon, Longboat Key, FL (US); Uma Mahesh Babu, Bradenton, FL (US)

(21) Appl. No.: 15/519,221

(22) PCT Filed: Oct. 8, 2015
(86) PCT No.: PCT/US15/54593

§ 371 Date: Apr. 14, 2017

Related U.S. Application Data
(60) Provisional application No. 62/064,164, filed on Oct. 15, 2014.
(60) Provisional application No. 62/064,137, filed on Oct. 15, 2014.
(60) Provisional application No. 62/064,151, filed on Oct. 15, 2014.
(60) Provisional application No. 62/065,911, filed on Oct. 20, 2014.
(60) Provisional application No. 62/065,920, filed on Oct. 20, 2014.
(60) Provisional application No. 62/065,935, filed on Oct. 20, 2014.

(51) Int. Cl. A61K 38/17; A61K 47/02; A61K 47/10; A61K 47/38; A61K 47/06

(52) U.S. Cl. CPC A61K 38/1709; A61K 47/38; A61K 47/06; A61K 47/10; A61K 47/02

(57) ABSTRACT

Histatins may be used for skin wound healing in humans and other animals. For example, histatins could be included in gels, ointments, creams, tissue glues, patches, aerosol sprays, subcutaneous injections, subcutaneous infusions, intradermal injections, intradermal infusions, or any combination of these formulations. In some embodiments, peptide fragments from at least two different histatins are used.

### REFERENCE TO RELATED APPLICATIONS

This application claims one or more inventions which were disclosed in Provisional Application No. 62/064,164, filed Oct. 15, 2014, entitled “FORMULATIONS FOR HISTATIN PROTECTIVES AND THERAPEUTICS”, Provisional Application No. 62/064,137, filed Oct. 15, 2014, entitled “FORMULATIONS FOR HISTATIN THERAPEUTICS”, Provisional Application No. 62/064,151, filed Oct. 15, 2014, entitled “FORMULATIONS FOR HISTATIN THERAPEUTICS”, Provisional Application No. 62/065,911, filed Oct. 20, 2014, entitled “FORMULATIONS FOR HISTATIN THERAPEUTICS” and Provisional Application 62/065,920, filed Oct. 20, 2014, entitled “FORMULATIONS FOR HISTATIN THERAPEUTICS” and Provisional Application No. 62/065,935, filed Oct. 20, 2014, entitled “FORMULATIONS FOR HISTATIN PROTECTIVES AND THERAPEUTICS”. The benefit under 35 USC §119(e) of the United States provisional applications is hereby claimed, and the aforementioned applications are hereby incorporated herein by reference.

### BACKGROUND OF THE INVENTION

Field of the Invention

The invention pertains to the field of wound and disease healing. More particularly, the invention pertains to epithelial wound healing and treating skin wounds using histatins.

Description of Related Art

Histatins have been shown in in vitro studies to be wound healing agents from saliva. More specifically, WO 2009/087117 (and its US equivalent US Patent Publication 2011/0178010), herein incorporated by reference, identified peptides of histatin, which had wound healing properties in vitro.

Histatin 1 (Hst-1) and Histatin 2 (Hst-2) have been identified as major wound-closing factors in human saliva (“Discovery of the Wound Healing Capacity of Salivary Histatins”, thesis of Menno Johannes Oudhoff, Academic Centre for Dentistry Amsterdam (ACTA), VU University Amsterdam and University of Amsterdam, The Netherlands, 2010, herein incorporated by reference). These studies were all done in vitro and cannot be translated to a finding for therapeutic or clinical use, especially since wound and disease healing are complex processes that need to be highly regulated in order to function properly.

### SUMMARY OF THE INVENTION

Histatins may be used for epithelial wound healing in humans and other animals. For example, histatins may be included in gels, ointments, creams, tissue glues, patches, aerosol sprays, subcutaneous injections, subcutaneous infusions, intradermal injections, intradermal infusions, or any combination of these formulations.

In one preferred embodiment, a method of treating skin wounds includes the step of administering a therapeutic amount of a peptide or a peptide fragment of at least two histatins at a site of a skin wound. The histatins are preferably administered using gels, ointments, creams, tissue glues, patches, aerosol sprays, subcutaneous injections, subcutaneous infusions, intradermal injections, intradermal infusions, or any combination of these formulations. In a preferred embodiment, the therapeutic amount of histatin accelerates wound healing compared to skin wounds not treated with histatin. The peptides or peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

In some preferred embodiments, the histatins include a) at least a peptide fragment of histatin 5 and b) at least a peptide fragment of histatin 1, at least a peptide fragment of histatin 2 or a combination of at least a peptide fragment of histatin 1 and at least a peptide fragment of histatin 2.

In other preferred embodiments, the histatin includes a) histatin 5 (SEQ ID NO: 30) and b) histatin 1 (SEQ ID NO: 4), histatin 2 (SEQ ID NO: 5), or a combination of histatin 1 and histatin 2. In other embodiments, the histatin comprises the amino acid sequence SEQ ID NO: 33 in a cyclized form. In yet other embodiments, the histatin includes a combination of peptide fragments of histatin 1, 2, and/or 5, and full length histatin 1, 2, and/or 5. In other preferred embodiments, the histatin includes histatin 5 (SEQ ID NO: 30), either in a cyclized or noncyclized form, and cyclized histatin 1 (SEQ ID NO: 33).

Histatin formulations for treating skin wounds include a first peptide comprising a first histatin selected from the group consisting of histatin 1, a fragment of histatin 1, histatin 2, a fragment of histatin 2, histatin 1 and histatin 2, histatin 1 and a fragment of histatin 2, histatin 2 and a fragment of histatin 1, or a fragment of histatin 1 and a fragment of histatin 2, and a second peptide comprising a second histatin selected from the group consisting of histatin 5 and a fragment of histatin 5. In some embodiments, the formulation includes approximately 50-100 μg/mL of a combined formulation of the first histatin and the second histatin. In some embodiments, the formulation includes 50-75 wt % of the first histatin and 25-50 wt % of the second histatin with respect to a total weight of histatins in the formulation. In some embodiments, the weight-to-weight ratio of histatin 1 to histatin 5 is 1:1, 2:1, 3:1, 4:1, 5:1, 1:2, 1:3, 1:4, or 1:5.

A method of treating a fungus, a virus, or a parasite on an epithelial surface includes the step of administering a therapeutic amount of at least one medicament comprising a first peptide and second peptide to the epithelial surface, wherein the first peptide comprises a first histatin or a fragment of the first histatin and the second peptide comprises a second histatin or a fragment of the second histatin.

### DETAILED DESCRIPTION OF THE INVENTION

Histatins are naturally occurring oral peptides produced by humans and non-human primates that demonstrate direct anti-infective activity, potent anti-inflammatory properties, and stimulate epithelial wound healing in several tissue and organ culture systems. A research facility has developed a technique to isolate this natural substance, making it a potential topical treatment for wounds.

“Wounds”, as defined herein, are injuries to living tissue, and can be caused by a cut, blow, or other impact. In most wounds, the skin or another external surface is cut or broken. Healing of those wounds using histatins 1 and 2 occurs by cell migration (epithelial migration, closing of an epithelial defect) and/or tissue regeneration. The regeneration occurs without induction of mitosis. In some embodiments, a wound is more specifically a physical manifestation of a breakdown of the protective function of the skin or other outer part of the body that normally provides a protective function, such as, for example, the cornea. In some embodiments, the wound reflects a loss of continuity of the epithelium. A cause of a wound may include, but is not limited to, surgery, a blow, a cut, contact with one or more chemicals, heat, cold, friction, a shear force, pressure, an ulcer, or a carcinoma.

As general protease inhibitors, histatins can overcome the deleterious effects of collagenases in skin. Since histatins are natural and general protease inhibitors, they inhibit the deleterious function of host's own elevated levels of proteases that appear in wound exudates such as collagenases and peptidoglycans and the proteases coming from the invading microbes such as toxins.

US Patent Publications 2013/0310326, and 2013/0310327, both published on Nov. 21, 2013, and entitled “HISTATIN FOR CORNEAL WOUND HEALING AND OCULAR SURFACE DISEASE”, and herein incorporated by reference, disclose histatins that may be used for corneal wound healing and as a treatment for ocular surface disease in humans and other animals. Ocular, nasal and oral surfaces are considered parts of the mucosal network of the individual. Skin is not a part of the mucosal network. The composition and the matrix properties of saliva (where histatins are naturally found) are very different from the wound cleansing solutions and wound healing antibiotic gels used on skin. Those skilled in the art would not find it obvious to use mucosal therapeutics to treat non-mucosal surfaces like skin lesions and wounds.

The histatins and the histatin sequences described herein may be used as anti-fungal agents, antiviral agents, and/or antiparasitic agents for the skin. In one embodiment, the histatins may be used to treat acanthamoeba skin infections. Acanthamoeba are one of the most common protozoa in soil, and can also be found in fresh water or other habitats.

The antiviral efficacy of a histatin may be verified through the following type of in vitro test: 150 μL of a solution containing one or more histatins and a control solution are aliquoted into separate sterile screw cap microfuge tubes. 150 μL of stock virus in phosphate-buffered saline (PBS) are added to each tube containing the compounds and are mixed. The drug containing and control tubes are then incubated at 37° C. At 60 minutes of incubation, 300 μL of fresh tissue culture medium containing 20% fetal bovine serum is added to the tubes. Standard viral plaque assays are immediately performed to determine the residual viral titers present in each sample. Viral titers (PFU+1) are Log₁₀ converted and Log₁₀ reductions in titers from the control are calculated for each trial. The mean±SD Log reduction in titer for each virus are calculated for the two trials. Mean reductions in titer of at least one Log₁₀ are considered effective reductions. Mean reductions in titer of three Log₁₀) (99.9%) are considered virucidal reductions.

The antiparasitic efficacy of a histatin may be verified through the following type of in vitro test: 0.1 mL of acanthamoeba inoculum is pipetted into 0.5 mL each of polyhexamethylene biguanide (PHMB) 0.02%, saline and two different concentrations of at least one histatin. The inoculated histatin and control samples are incubated at 30° C. for 24 hours. At 24 hours, 0.05 mL of the inoculated samples is removed from the mixtures and plated on non-nutrient agar overlaid with Enterobacter aerogenes using a glass rod to disperse the samples. This prevents a concentrated amount of histatin to inhibit bacterial growth. The overlay is prepared by spreading of 0.3 mL of the Enterobacter aerogenes slurry on a non-nutrient agar with a soft-tipped applicator. The plates are incubated at 30° C. in an air incubator. After another 24 hours, a second overlay of Enterobacter aerogenes is administered to assure the food source is available to the acanthamoeba without any effect of residual drug. All plates are monitored for the robust growth of acanthamoeba resulting in a mixture of sparse trophozoites and predominant cysts at days 7 and 14. Robust growth at day 7 terminates testing with a positive result. At day 14, all remaining plates (plates observed for the lack of robust growth) are vigorously sub-cultured with a soft-tipped applicator onto fresh non-nutrient agar overlaid with Enterobacter aerogenes. After 7 days incubation, all plates are monitored for the robust growth of acanthamoeba resulting in a mixture of sparse trophozoites and predominant cysts. For each drug concentration, positive growth is graded as a “1”, and no growth is denoted as “0”. Any incidence of positive growth after 24 hours is considered as survival. Negative growth at 24 hours is denoted as “kill”.

In a preferred embodiment, a peptide including at least one amino acid sequence of at least eight amino acids adjacently present in Histatin 1, 2, 3, and/or 5 is used to treat an epithelial or skin wound. In other preferred embodiments, multiple histatin peptides or peptide fragments are used.

In one preferred embodiment, a method of treating skin wounds includes the step of administering a therapeutic amount of at least a portion of a histatin peptide at a site of a skin wound. In another preferred embodiment, a method of treating skin wounds includes the step of administering a therapeutic amount of at least a portion of at least two histatin peptides at a site of a skin wound.

The histatins are preferably administered using gels, ointments, creams, tissue glues, patches, aerosol sprays, subcutaneous injections, subcutaneous infusions, intradermal injections, intradermal infusions, or any combination of these formulations. In a preferred embodiment, the therapeutic amount of histatin accelerates healing compared to skin wounds not treated with histatin.

In some preferred embodiments, the histatin concentration is between approximately 0.1 μg/mL and approximately 1000 mg/mL. In other preferred embodiments, the histatin concentration is between approximately 0.1 μg/mL and 100 μg/mL. In other preferred embodiments, the histatin concentration is between approximately 0.1 μg/mL and 10 μg/mL. In some preferred embodiments, the histatin concentration is greater than or equal to approximately 1 μM.

The administering step may be repeated multiple times per day and/or for a plurality of days. In one preferred embodiment, this step is repeated at least one time a day for a plurality of days. In another preferred embodiment, the step is repeated chronically at least one time a day. In some preferred embodiments, the step is repeated up to hourly for a plurality of days. In another preferred embodiment, the step is repeated at least two times a day for a plurality of days. In yet another preferred embodiment, the step is repeated at least three times a day for a plurality of days, for example for seven days. In another preferred embodiment, the step is repeated four times a day for five days.

In one preferred embodiment, at least one of the histatins is a peptide including 8 to 44 amino acids. In some preferred embodiments, at least one of the peptides is an L-peptide. In other preferred embodiments, at least one of the peptides is a cyclic peptide.

In some preferred embodiments, the amino acid sequence of the histatin peptide is one or more of SEQ ID NOS: 1 through 33, or any combinations of these sequences. In alternative embodiments, one or more of the amino acid sequences have a substitution, deletion and/or insertion of up to 3 amino acids. In other alternative embodiments, one or more of the amino acid sequences have a substitution, a deletion and/or an insertion of two or less amino acids. In other alternative embodiments, one or more of the amino acid sequences have a substitution, a deletion, and/or an insertion in one amino acid.

The SEQ ID NO: 4 peptide is also known as Histatin 1 (Hst-1). Note that the first serine in this amino acid sequence may be a phosphoserine. The SEQ ID NO: 5 peptide is also known as Histatin 2 (Hst-2, also equivalent to amino acids 12-38 of Hst-1). The SEQ ID NO: 6 peptide is also known as Histatin 3 (Hst-3). The SEQ ID NO: 30 peptide is also known as Histatin 5 (Hst-5). Parts and fragments of each of these amino acid sequences may be used, alone or in combination, including but not limited to SEQ ID NOS: 1-3, 7-29 (for Histatin 1, Histatin 2 and Histatin 3) and SEQ ID NO: 32 (for Histatin 5) to facilitate wound closure in the embodiments described herein. While the L stereoisomer of the amino acids is preferred for the amino acid sequences described herein, D stereoisomers may alternatively be used. Alternatively, amino acid sequences that include these histatins and other amino acids, for example SEQ ID NO: 33, which is a sortase cyclized histatin (including all of Histatin 1), may be used in the embodiments described herein. Any histatin sequences could be cyclized and used in the embodiments described herein.

In one preferred embodiment, a method of treating skin wounds includes the step of administering a therapeutic amount of at least a peptide fragment of at least two histatins at a site of an epithelial wound. The histatins are preferably administered using gels, ointments, creams, tissue glues, patches, or any combination of these formulations. In preferred embodiments, using histatin amino acid sequences that promote skin wound closure (for example, a histatin amino acid sequence present in histatin 1 or histatin 2) in combination with a histatin amino acid sequence with antimicrobial properties (for example, a histatin amino acid sequence present in histatin 5), has synergistic healing effects. This strategy combines the direct effects that histatin 1 and histatin 2 have on wound closure with the indirect effects the antimicrobial properties of histatin 5 have on wound closure. More specifically, histatins 1 and 2 promote wound closure, while histatin 5 prevents microbial infection, thereby creating a better environment for wound healing.

In a preferred embodiment, the therapeutic amount of histatin accelerates wound healing compared to skin wounds not treated with histatin. The peptide fragments of the histatins preferably includes at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

Some preferred embodiments use amino acid sequences from Hst-1 and/or Hst-2 in combination with amino acid sequences from Hst-5 to treat skin wounds. In these embodiments, one or more amino acid sequences from Hst-1 and/or Hst-2 are chosen, and one or more amino acid sequences from Hst-5 are chosen. In some embodiments, the full length Histatin 1 (SEQ ID NO: 4), full length Histatin 2 (SEQ ID NO: 5), and/or the full length Histatin 5 (SEQ ID NO: 30) could be used. In other embodiments, portions of Hst-1, Hst-2, and/or Hst-5 could be used. For example, SEQ ID NO: 29, which is equivalent to amino acids 20-32 of Histatin 1, may be a preferred amino acid sequence to use for wound closure in some embodiments. In other examples, peptides including SEQ ID NO: 32, a peptide fragment of Histatin 1 and Histatin 2 that appears to be a core motif for wound closure, may be used. Other preferred sequences from Hst-1 and Hst-2 include, but are not limited to, SEQ ID NO: 8, SEQ ID NO: 9 and SEQ ID NO: 13. As another example, SEQ ID NO: 31, a fragment of Histatin 5 (Gusman et al., “Salivary Histatin 5 is an inhibitor of Both Host and Bacterial Enzymes Implicated in Periodontal Disease”, Infect. Immun. 2001, 69(3): 1402, pp. 1402-1408, herein incorporated by reference), may be used, preferably in combination with Histatin 1 or Histatin 2 or fragments thereof. In other preferred embodiments, fragments of Hst-1 or Hst-2 are used with full length Hst-5 (SEQ ID NO: 30) or full length Hst-1 (SEQ ID NO: 4) or Hst-2 (SEQ ID NO: 5) are used with fragments of Hst-5 (for example, SEQ ID NO: 31). In yet other embodiments, any combination of fragments of Hst-1 and/or Hst-2, full length Hst-1 and/or Hst-2, fragments of Hst-5, or full length Hst-5 may be used. In some preferred embodiments, the concentration of the Hst-5 peptide used is greater than or equal to approximately 1 μM.

The amino acids and the peptides described herein may include at least one functional grouping (for example, an amine and/or carboxylic group) protected with a protective grouping in some embodiments. Since the peptides are applied to tissue, skin or a wound, a protected form of the peptide may be preferred to resist degradation. The form of protection needs to be biologically compatible and compatible with pharmaceutical use. Some examples include, but are not limited to, the acylation or the acetylation of the amino-terminal ends, cyclization or the amidation or the esterfication of the carboxy-terminal ends. Thus, the peptides described herein may be used in a protected form.

The peptides described herein may be made by traditional chemical synthesis, enzymatic synthesis, or any other method known in the art.

The peptides preferably include at least 8 amino acids. In one preferred embodiment, the peptides include a range of 8 to 44 amino acids, but the peptides may alternatively include more than 44 amino acids.

Histatins and peptide portions or peptide fragments of histatins may be used to accelerate skin wound healing in humans and other animals. In preferred embodiments, histatin 1 (Hst-1), histatin 2 (Hst-2), histatin 5 (Hst-5), peptide fragments of Hst-1, Hst-2, or Hst-5, or any combinations thereof may be used. In other embodiments, histatin 3 (Hst-3) or the D-enantiomer of histatin 2 (D-Hst-2), or peptide fragments thereof, may be used. Any combinations of any of the histatins may be used. In preferred embodiments, histatin concentrations between 0.1 μg/mL and 1000 mg/mL may be used. Peptides with amino acid SEQ ID NOS: 1-33, histatins known in the art, the peptides disclosed in WO 2009/087117 or the peptides disclosed in Dr. Menno Johannes Oudhoff's thesis, “Discovery of the Wound-Healing Capacity of Salivary Histatins”, 2010, department of Oral Biochemistry of the Academic Centre for Dentistry Amsterdam (ACTA), VU University Amsterdam and University of Amsterdam, The Netherlands, herein incorporated by reference, may be used.

In one preferred embodiment, histatin 1 (Hst-1) or histatin 2 (Hst-2) in combination with histatin 5 (Hst-5), peptide fragments of Hst-1 or Hst-2 in combination with peptide fragments of Hst-5, or any combination, are used. Hst-5 inhibits production of Matrix Metalloproteases (MMPs).

The combination of the Hst-1/Hst-2 healing properties with the Hst-5 inhibiting MMPs should be very effective. In some preferred embodiments, a concentration of at least approximately 1 μM of Hst-5, or a fragment of Hst-5, is used.

In one preferred embodiment, a cyclic version of SEQ ID NO: 33 is used in combination with SEQ ID NO: 30, either in a cyclized or non-cyclized form.

Histatins could be administered to humans or other animals with a skin wound. Some methods of administration include, but are not limited to, incorporating the histatin into gels, ointments, creams, tissue glues (to transiently seal skin injuries), patches, aerosol sprays, subcutaneous injections, subcutaneous infusions, intradermal injections, intradermal infusions, or combinations of these formulations.

The histatins may be administered in any combination of daily treatments for any number of days in order to produce therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

Histatin Formulations for Skin Wounds

In some embodiments, a formulation for histatin for skin wounds includes a total of approximately 25-150 μg/mL of a combination of a histatin 1 fragment and a histatin 5 fragment in an applying vehicle. In some preferred embodiments, a formulation for histatin for skin wounds includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation. In some preferred embodiments of this formulation, both the histatin 1 and the histatin 5 fragment are cyclized. In other preferred embodiments, one of the fragments is cyclized. In other embodiments, neither of the fragments is cyclized.

In one preferred embodiment, the histatin 1 fragment is a cyclized version of SEQ ID NO: 33. In another preferred embodiment, the histatin 5 fragment is SEQ ID NO: 30. In another preferred embodiment, the histatin 5 fragment is a cyclized version of SEQ ID NO: 30. In another preferred embodiment, the histatin 1 fragment is a cyclized version of SEQ ID NO: 33 and the histatin 5 fragment is SEQ ID NO: 30 (either in a cyclic or linear form).

In other preferred embodiments, the histatin formulation comprises only Histatin 1. In some of these embodiments, the histatin 1 fragment is a cyclized version of SEQ ID NO: 33.

In some embodiments, the preferred vehicle in histatin formulations for the skin is a mixture of 50 wt %-65 wt % white petrolatum and 35 wt %-50 wt % purified water with 2 wt %-2.5 wt %, preferably 2.3 wt %, of a modified carboxymethycellulose polymer or liquid paraffin together with ethylene glycol, aloe, or propylene glycol.

Other alternative or additional ingredients include, but are not limited to, liquid paraffin, ethylene glycol, aloe barbadensis (aloe vera) gel, monostearate, stearic acid, paraffin wax, aluminum sulfate, calcium acetate, cetearyl alcohol, mineral oil, maltodextrin, white wax, and any combination of these ingredients.

Some preferred preservatives to be used in the formulation include, but are not limited to, potassium sorbate, propylparaben, Benzalkonium chloride (BAK), or any combination of these preservatives.

Metal ions and metal suspensions such as silver and colloidal gold have been used in sprays, emulsions etc. for their antimicrobial activity. There are therapeutic silver ointments and gels that are used to cleanse and treat wounds. Zinc lozenges (for example, Zicam® zinc lozenges) are used to lessen the duration of flu. In some embodiments, a metal ion or a metal suspension including, but not limited to, silver, colloidal gold, or zinc, may be included in the histatin skin formulations.

These formulations could be administered to humans or other animals with a skin wound. Some methods of administration include, but are not limited to, incorporating the histatin into gels, ointments, creams, tissue glues (to transiently seal skin injuries), patches, aerosol sprays, subcutaneous injections, subcutaneous infusions, intradermal injections, intradermal infusions, or any combination of these formulations. In one preferred embodiment, the histatin formulation is between an ointment and a cream in physical properties.

These formulations may be administered in any combination of daily treatments for any number of days in order to produce therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

All of the patent and nonpatent references discussed herein are incorporated herein by reference.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A histatin formulation for treating skin wounds comprising:
a) a first peptide comprising a first histatin selected from the group consisting of:
i) histatin 1;
ii) a fragment of histatin 1;
iii) histatin 2;
iv) a fragment of histatin 2;
v) histatin 1 and histatin 2;
vi) histatin 1 and a fragment of histatin 2
vii) histatin 2 and a fragment of histatin 1;
viii) a fragment of histatin 1 and a fragment of histatin 2; and
b) a second peptide comprising a second histatin selected from the group consisting of:
i) histatin 5; and
ii) a fragment of histatin 5;
wherein the formulation comprises approximately 50-100 μg/mL of a combined formulation of the first histatin and the second histatin.

2. The formulation of claim 1, wherein the formulation comprises 50-75 wt % of the first histatin and 25-50 wt % of the second histatin with respect to a total weight of histatins in the formulation.

3. The formulation of claim 1, wherein a weight-to-weight ratio of histatin 1 to histatin 5 is selected from the group consisting of: 1:1, 2:1, 3:1, 4:1, 5:1, 1:2, 1:3, 1:4, 1:5 29.

4. The formulation of claim 1, wherein at least one of the first histatin and the second histatin is cyclized.

5. The formulation of claim 1, wherein the formulation comprises SEQ ID NO: 30 and SEQ ID NO: 33.

6. The formulation of claim 1, further comprising a vehicle mixture of 50 wt %-65 wt % white petrolatum and 35 wt %-50 wt % purified water.

7. The formulation of claim 6, further comprising a) 2.3 wt % of a modified carboxymethycellulose polymer or b) liquid paraffin with ethylene glycol, aloe, or propylene glycol.

8. The formulation of claim 1, further comprising a) 2.3 wt % of a modified carboxymethycellulose polymer or b) liquid paraffin with ethylene glycol, aloe, or propylene glycol.

9. The formulation of claim 1, further comprising a preservative selected from the group consisting of: potassium sorbate, propylparaben, Benzalkonium chloride (BAK), and any combination of these preservatives.

10. The formulation of claim 1, further comprising a metal ion or a metal suspension.

11. A histatin formulation for treating skin wounds comprising:
a) a first peptide comprising a first histatin selected from the group consisting of:
i) histatin 1;
ii) a fragment of histatin 1;
iii) histatin 2;
iv) a fragment of histatin 2;
v) histatin 1 and histatin 2;
vi) histatin 1 and a fragment of histatin 2;
vii) histatin 2 and a fragment of histatin 1;
viii) a fragment of histatin 1 and a fragment of histatin 2; and
b) a second peptide comprising a second histatin selected from the group consisting of:
i) histatin 5; and
ii) a fragment of histatin 5;
wherein the formulation comprises 50-75 wt % of the first histatin and 25-50 wt % of the second histatin with respect to a total weight of histatins in the formulation.

12. The formulation of claim 11, wherein at least one of the first histatin and the second histatin is cyclized.

13. The formulation of claim 11, wherein the formulation comprises SEQ ID NO: 30 and SEQ ID NO: 33.

14. The formulation of claim 11, further comprising a vehicle mixture of 50 wt %-65 wt % white petrolatum and 35 wt %-50 wt % purified water.

15. The formulation of claim 14, further comprising a) 2.3 wt % of a modified carboxymethycellulose polymer or b) liquid paraffin with ethylene glycol, aloe, or propylene glycol.

16. The formulation of claim 11, further comprising a) 2.3 wt % of a modified carboxymethycellulose polymer or b) liquid paraffin with ethylene glycol, aloe, or propylene glycol.

17. The formulation of claim 11, further comprising a preservative selected from the group consisting of: potassium sorbate, propylparaben, Benzalkonium chloride (BAK), and any combination of these preservatives.

18. The formulation of claim 11, further comprising a metal ion or a metal suspension.

19. A histatin formulation for treating skin wounds comprising
a) the cyclized sortase histatin of SEQ ID NO: 33;
b) a vehicle mixture of 50 wt %-65 wt % white petrolatum and 35 wt %-50 wt % purified water; and
c) 2.3 wt % of a modified carboxymethycellulose polymer or b) liquid paraffin with ethylene glycol, aloe, or propylene glycol.

20. The formulation of claim 19, further comprising a preservative selected from the group consisting of: potassium sorbate, propylparaben, Benzalkonium chloride (BAK), and any combination of these preservatives.

21-57. (canceled)
